(19) United States
(12) Patent Application Publication
(10) Pub. No.: US 2018/0260912 A1
(43) Pub. Date: Sep. 13, 2018

(54) SOCIAL NETWORKING SYSTEM FOR THE MILITARY COMMUNITY

(71) Applicant: Alyssa Quinn, Newall, IA (US)

(72) Inventor: Alyssa Quinn, Newall, IA (US)

(21) Appl. No.: 15/916,626

(22) Filed: Mar. 9, 2018

Related U.S. Application Data
(60) Provisional application No. 62/469,279, filed on Mar. 9, 2017.

(51) Int. Cl. G06Q 50/00; H04W 4/021; G06F 17/30; G16H 20/70

(52) U.S. Cl. CPC G06Q 50/01; G16H 20/70; G06F 17/30867; H04W 4/021

(57) ABSTRACT

A social networking system for veterans, veteran owned businesses, and businesses that offer products and services to the veteran community. An embodiment of a social networking system includes providing a social networking system, gathering information about the users' military history, verifying the users' military affiliation, and providing information to the one or more users.

### CROSS-REFERENCE TO RELATED APPLICATIONS

This application claims priority to U.S. Provisional Patent Application No. 62/469,279, filed on Mar. 9, 2017, the entirety of which is hereby incorporated herein by reference.

### FIELD OF THE INVENTION

This invention relates generally to the field of social networking, and more specifically to providing a social networking system for the military community.

### BACKGROUND

The veteran suicide rate is increasing at a devastating pace. According to the Veteran's Administration (VA), approximately 20 veterans commit suicide daily, and many more have issues with substance abuse. Veterans struggle to find help for mental health and substance abuse problems.

Certain web sites and mobile applications provide services to the veteran community. Meet up and Rally Point are two web sites that offer articles and networking for veterans, but they are not user friendly, and are not widely used by the veteran community. The VA has websites to show available resources to veterans, but they are difficult to maneuver, and are not very popular with the veteran community. Other sites, such as VOBN.com (Veteran Owned Business directory) show many veteran owned businesses, but only offers that information.

The existing resources and sources of information have not addressed the rising veteran suicide rate. Thus, a comprehensive and easily navigable mobile application or web site for the veteran community for networking, support, interaction, and access to services, nearby events, and veteran businesses that is appealing to the veteran community is needed.

### BRIEF SUMMARY

In accordance with one embodiment of the invention, a method of providing information to the veteran community is disclosed. By providing a comprehensive source of information for veterans and current service members, the rising rate of veteran suicide is addressed by helping veterans locate other veterans, getting veterans involved in local events and contributing back to the veteran community by being involved in these events, and providing access to resources without users having to search large databases or read through a great deal of content before realizing a specific service may not even be available to them.

Information is provided via a mobile application and a website, and includes a comprehensive resource guide for current service members and those leaving the service, with a strong emphasis on mental health, substance abuse, and job placement outside of the military. Information about how to access other veterans nearby, events happening in their area, and searchable lists of local and national veteran owned businesses as well as businesses that offer discounts for services and products to the military community are also provided.

Through geo-tagging, veterans are able to locate people, events, and available resources near them. Veterans are able to locate other veterans with similar military backgrounds and interests. An events calendar for the veteran community is provided based on a number of interests (competitive fitness events, shooting, outdoor activities, etc.) Veterans may access local ambassadors to assist them while traveling through particular areas. The Red Flag initiative feature of the app is used to notify other veterans users of the app that the veteran who has activated the feature is in a crisis moment and they are seeking peer support from other Veterans nearby. Simple and easy-to-use resource guides to assist current service members and veterans throughout their military career and after are provided. A searchable database of veteran owned businesses is also provided in order to encourage veteran business networking as well as support veteran entrepreneurs. Non-profit organizations that provide services to the veteran community are featured, links to veteran job placement sites are provided, and companies that offer discounted rates for products and services to the veteran community are also showcased.

In other embodiments of the invention, information and services for veterans' family members (spouses, kids and survivors of the fallen), the civil support community (fire, law, first responders, etc.), and members of the armed forces of other nations are provided.

The invention provides a single-source solution for networking, support, interaction, and access to resources for the military community. The user interface is creative and engaging, with user friendly functionality

### BRIEF DESCRIPTION OF THE DRAWINGS

Having thus described the invention in general terms, reference will now be made to the accompanying drawings, which are not necessarily drawn to scale, and wherein:

FIG. 1 illustrates a system for providing information to the military community in accordance with an embodiment of the invention.

FIG. 2 illustrates a method for providing information to the military community in accordance with an embodiment of the invention.

FIG. 3 illustrates a user interface in accordance with an embodiment of the invention.

FIG. 4 illustrates a user interface in accordance with an embodiment of the invention.

FIG. 5 illustrates a user interface in accordance with an embodiment of the invention.

FIG. 6 illustrates a user interface in accordance with an embodiment of the invention.

FIG. 7 illustrates a user interface in accordance with an embodiment of the invention.

FIG. 8 illustrates a user interface in accordance with an embodiment of the invention.

FIG. 9 illustrates a user interface in accordance with an embodiment of the invention.

FIG. 10 illustrates a user interface in accordance with an embodiment of the invention.

FIG. 11 illustrates a user interface in accordance with an embodiment of the invention.

FIG. 12 illustrates a user interface in accordance with an embodiment of the invention.

FIG. 13 illustrates a user interface in accordance with an embodiment of the invention.

FIG. 14 illustrates a user interface in accordance with an embodiment of the invention.

FIG. 15 illustrates a method for alerting users of a social networking system that another user is experiencing a crisis situation in accordance with an embodiment of the invention.

FIG. 16 illustrates a user interface for alerting users of a social networking system that another user is experiencing a crisis situation in accordance with an embodiment of the invention.

### DETAILED DESCRIPTION

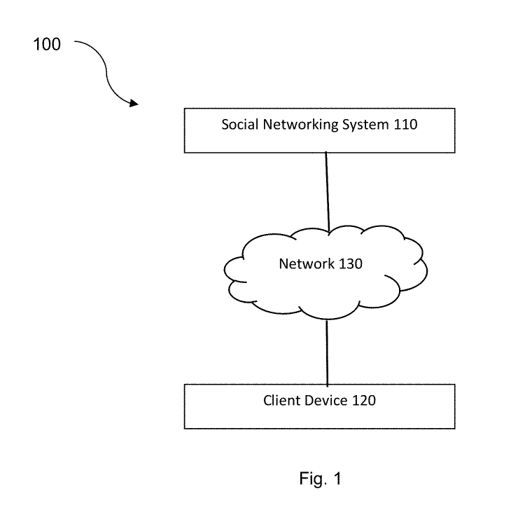

In accordance with one embodiment of the invention, a system for providing information to the veteran community 100 is shown in FIG. 1. The system 100 comprises a social networking system 110 and at least one client device 120. The social networking system 110 and the client device 120 are connected by a network 130.

Social networking system 110 includes one or more computing devices storing user profiles, connections between users, and information for users. Users register to use the social networking system 110, and may then add connections to other users of the social networking system 110 or view information stored on the networking system.

Users view content made available through the social networking system 110 using a client device 120. Using an input device (i.e. keyboard, mouse, touchscreen, or other input device), users may enter information to be transmitted to the social networking system 110. Client device 120 may comprise a mobile phone, tablet, laptop, personal computer, or any other device capable of accessing information over network 130.

Information is transferred between client device 120 and social networking system 110 via a network 130. Network 130 can include any combination of local area and/or wide area networks, using both wired and wireless communication systems. In one embodiment, the network 130 uses standard communications technologies and/or protocols.

In one embodiment, the client device 120 executes an application allowing a user of the client device 120 to interact with the social networking system 110. For example, the client device 120 may execute a browser application to enable interaction between the client device 120 and the social networking system 110 via the network 130. In another embodiment, a client device 120 interacts with the social networking system 110 through an application programming interface (API) that runs on the native operating system of the client device 120.

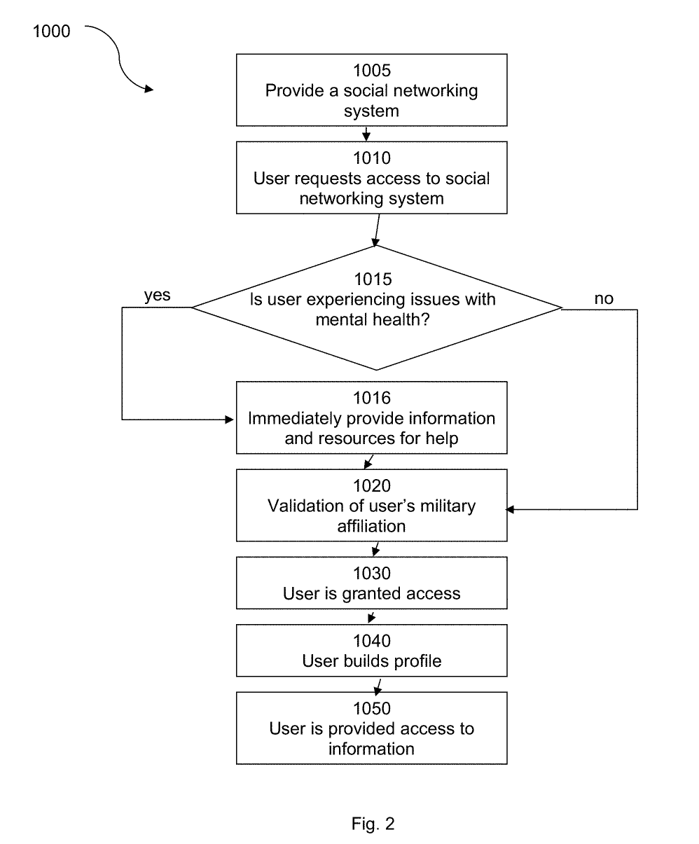

As shown in FIG. 2, in accordance with one embodiment of the invention, a method of providing information to the veteran community 1000 begins with step 1005 in which a social networking system 110 for providing information is provided. Certain information may provided to all users regardless of whether they have registered to use the social networking system 110. For example, contact information for suicide hotlines and other mental health resources may be provided to all visitors. In addition, information about featured veteran owned businesses and businesses that offer discounted rates to veterans may be provided to all visitors.

In step 1010, using a client device 120 a user requests access to restricted parts of the social networking system 110 by supplying identifying information, including name, branch of service, and military identification.

At step 1015, the social networking system 110 queries if the user is experiencing issues with mental health, such as thoughts of suicide or substance abuse. If the user answers “yes,” then the social networking system 110 immediately provides the user with information and available resources at step 1016, such as crisis hotline phone numbers before the method proceeds to step 1020. The resources may include contact information for facilities in the user's geographic area that can provide treatment for mental health issues, substance abuse, or other concerns. If the user answers “no,” then the method proceeds to step 1020.

In step 1020, the user's military affiliation is validated by social networking system 110. The user's military affiliation may be validated through the use of a third party verification service, such as ID.me, which is the same company USAA and the VA utilize. Through the use of the user's social security number, DD-214, or current membership to that third party's verification service. If the user is confirmed to be a veteran or current service member, the user is granted access to social networking system 110 at step 1030, and can access the social networking system 110 using a unique user name and password.

Once the user is granted access to the social networking system 110, the user may build his or her profile in step 1040. The step of building a profile 1040 comprises completing a military background questionnaire. Answers to the questions in the military background questionnaire are used to better connect the user with veterans close by who may have had similar experiences in the military and combat. The user's location is determined through geo-tagging in which the application or website automatically determines the user's location, or the user may manually enter his or her zip code, city and state, or other indication of geographic location using an input device associated with client device 120. The military background questionnaire may ask which branch of service the user is or was in (Army, Navy, Marines, Coast Guard, Air Force, Active/Reserve/National Guard), the years the user served, which military occupation codes/military occupational specialty codes (MOS's) the user has held, the duty stations the user has been assigned to and timelines, where the user has deployed to and timelines. The military background questionnaire may also ask if the user has any significant awards or experiences he or she would like to share in order to better connect the user with other veterans, to which the user may input purple heart recipient, navy seal, Airborne, or other significant awards or experiences. The step of building a profile 1040 may also include a personal interest questionnaire that is used to determine what the user is seeking in his or her connections, and to better connect the user with veterans with similar interests nearby. The personal interest questionnaire may ask the user what they are looking for in a connection, and the user may indicate their personal interests. The personal interests that the user can select or manually enter may include “buddies for hanging out” with a manually entered brief description of what the user likes to do; “buddies for fitness” with a description of preferred activities such as weight lifting, power lifting, cardio, triathlons, marathons, MMA, yoga, swimming, training, or other manually input activity; “buddies for drinking” with a description of the type of alcohol (i.e. liquor, beer, or wine) from a drop-down menu and indication of which establishments the user likes to visit; “study buddies” with a user selected or manually entered school and program of study; “buddies for shooting,” with a description of what the user likes to shoot and where; “buddies for building” with a user selected or manually entered description of what the user likes to build (i.e. homes, cars, auto repair, guns, crafts, woodwork, or other manually entered option); “buddies for families” with indication of whether the user is married, has children, and the gender and ages of the user's spouse and children; “buddies for religion” with user selected or manually entered description of the user's religion, where they practice their religion, and whether the user is affiliated with any religious groups; “buddies for art” with a user selected or manually entered description of the type of art (i.e. graphic design, painting, drawing, photography, multi-media, or other art form); “buddies for gaming” with a user selected or manually entered description of the type of gaming system the user uses and games the user likes to play, including board and card games; “buddies for volunteering” with a description of the type of volunteer work the user does and where; or other personal activities that users may want to do with others. The step of building a profile 1040 may also include a prompt asking the user if he or she would like to be a local ambassador for his or her area. A local ambassador is a veteran who lives in a particular area that is willing to share information with other veterans to help those passing through that area. If the user elects to become a local ambassador, the user fills out a short questionnaire that populates a personal biography that is displayed for other users seeking a local ambassador. The local ambassador questionnaire may ask for the user's name, branch of service, places they like to go, places to avoid, good gyms in the area, places to eat, places to stay, and other information about the local ambassador's geographic area. The local ambassador biography may also include an option to connect with and send direct messages to the local ambassador.

After building a profile, using a client device 120 the user can access information from the social networking system 110 at step 1050. The information provided to registered users includes information about other veterans or service members in the user's geographic area that have similar military backgrounds or interests, and the user may connect with them. The information may also include information on suicide awareness and hotlines, information on substance abuse and self-help (i.e. how to self-report, what to expect, rights and restrictions, regulations regarding substance abuse, etc.), job placement resources for veterans, upcoming events hosted by veterans in the user's geographic area (i.e. American Legion hosted charity events or motorcycle rides in a particular area), quick links to available resources, local ambassadors to assist veterans who are traveling to new areas, military pay charts, current regulations and forms for each branch of service, information regarding retirement from the military, health care, financial assistance, legal information and assistance, SHARP (sexual harassment and assault response and prevention) information for victims and accused/offenders), advertising for veteran owned businesses, advertising for other businesses that cater to or provide discounts to military members and veterans, non-profit organizations that provide services to veterans, and other information relevant to the user. Resources available to the military community will be featured with a quick, easy-to-read, and understandable description of each resource, “how-to” guides for using available resources, and links to get more information regarding those services (i.e. services provided by the VA (i.e. vocational rehabilitation, educational benefits, disability, and enrollment), suicide and mental health information, substance abuse assistance, financial and legal information and links to helpful resource sites, SHARP information and available resources, etc.) The user will be able to easily navigate through the user interface 200 of social networking system 110 to locate veteran owned and civilian owned businesses near them either by searching for specific criteria or simply browsing through the list. Veteran feedback on the businesses may determine their placement on the lists; for example, a business that does not supply the products they claim to, or not to the caliber they have promised; may still be featured but will fall lower on the list.

In addition to accessing information, the user may also post information for other users to access. Because the social networking system 110 caters to the military community, certain information cannot be included on the social networking system 110. For example, the Armed Forces Disciplinary Control Board maintains a list of “black listed” locations or websites that are banned to all military members. The list changes several times a year. The list is made by law enforcement and military installation, and the Armed Forces Disciplinary Control Board makes the final determination of whether an establishment or website should be black listed. To comply with the requirement that black listed locations and websites not be included, user-initiated posts of information to the social networking system 110 may be compared to the Armed Forces Disciplinary Control Board before they can be displayed to others via the social networking system 110. Examples of activities that would be acceptable to post may include: group fitness activities, group drinking activities, parties or gatherings at a specific location or business, events for specific hobbies (i.e. motorcycle rides, horse riding lessons, auto repair groups, competitive shooting or shooting for leisurely activity, religious gatherings), non-profit, charity, or volunteer events, or support at military funerals. Examples of activities that would be blocked from being posted using the social networking system 110 may include: any event that is being held at a black listed location, any event which encourages the use of illegal substances, any event tied to extremist groups or activities, any event which violates the Warrior Ethos, barracks parties, or events which exclude specific groups (i.e. by race, gender, or nationality).

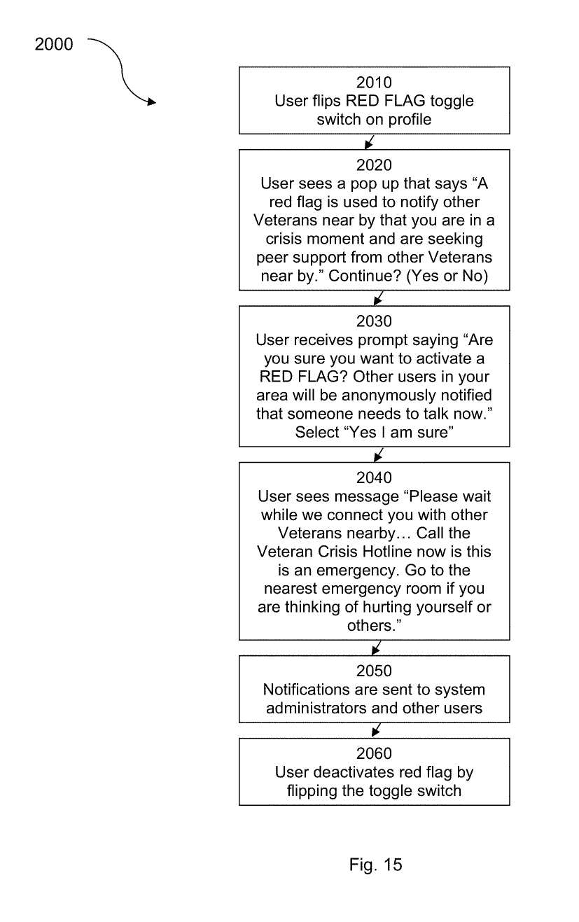

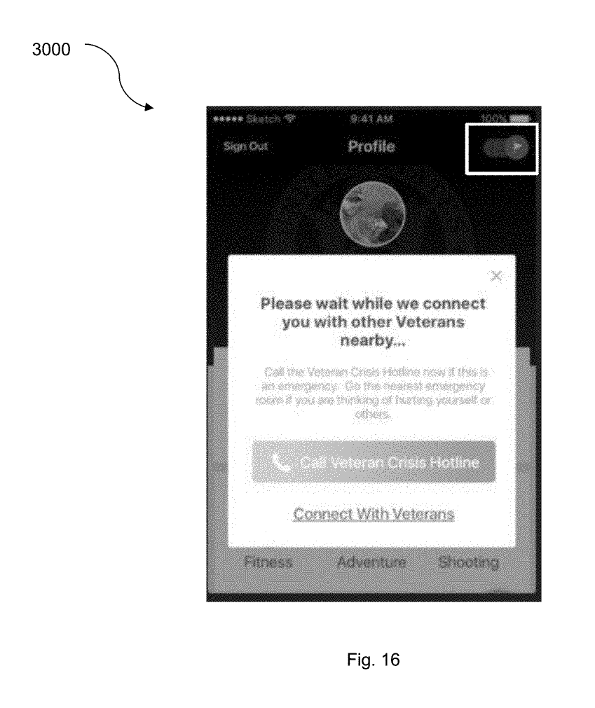

Any user who has been granted access to use the social networking system 110 can activate a Red Flag. The Red Flag initiative feature of the social networking system 110 comprises a notification system that is used to notify other users that the veteran who has activated the Red Flag feature is in a crisis moment, and they are seeking peer support from other Veterans nearby. As shown in FIG. 15, a method for initiating a Red Flag 2000 begins at step 2010 when a user flips the RED FLAG toggle switch on his or her profile. Only the user can initiate a Red Flag for themselves. At step 2020, the user sees a pop up on the screen of his or her client device 120 that says “A red flag is used to notify other Veterans nearby that you are in a crisis moment and are seeking peer support from other Veterans nearby.” Continue? (Yes or No). If the user selects “Yes,” then the method proceeds to step 2030 in which the user receives a prompt saying “Are you sure you want to activate a RED FLAG? Other users in your area will be anonymously notified that someone needs to talk now.” If the user selects “Yes I am sure”, then a Red Flag is activated, and the method proceeds to step 2040. At step 2040, the user sees the message “Please wait while we connect you with other Veterans nearby. . . Call the Veteran Crisis Hotline now is this is an emergency. Go to the nearest emergency room if you are thinking of hurting yourself or others.” At step 2050, the social networking system 110 sends notifications of the Red Flag situation via the network 130. All administrators of the social networking system 110 receive an alert with the name and location of the user who activated the Red Flag. All users of the social networking system 110 within 200 miles of the user who activated the Red Flag receive a notification saying “Someone near you has activated a RED FLAG. Would you like to connect with them?”(Yes or No). If a nearby user selects “yes”, then the nearby user can connect and message. Any nearby user who selects “no” will not know who initiated the Red Flag. At step 2060, the user who initiated the Red Flag may deactivate the Red Flag by again flipping the RED FLAG toggle switch on his or her profile. The social networking system 110 prompts the user with “Are you sure you want to deactivate your RED FLAG? If the user selects, :yes”, then the social networking system displays the message “We're glad you're feeling better!” on the screen of the user's client device 120, and the Red Flag is deactivated. A user interface 3000 for the Red Flag features is illustrated in FIG. 16.

The social networking system 110 may feature a selected “military member of the month” showcasing a member or veteran of the Armed Services who has been discharged honorably and has contributed to society in a significant way, or a “veteran owned business of the month.” The social networking system 110 may also feature articles, answers to questions frequently asked by the military community, news, or entertainment relevant to the military community.

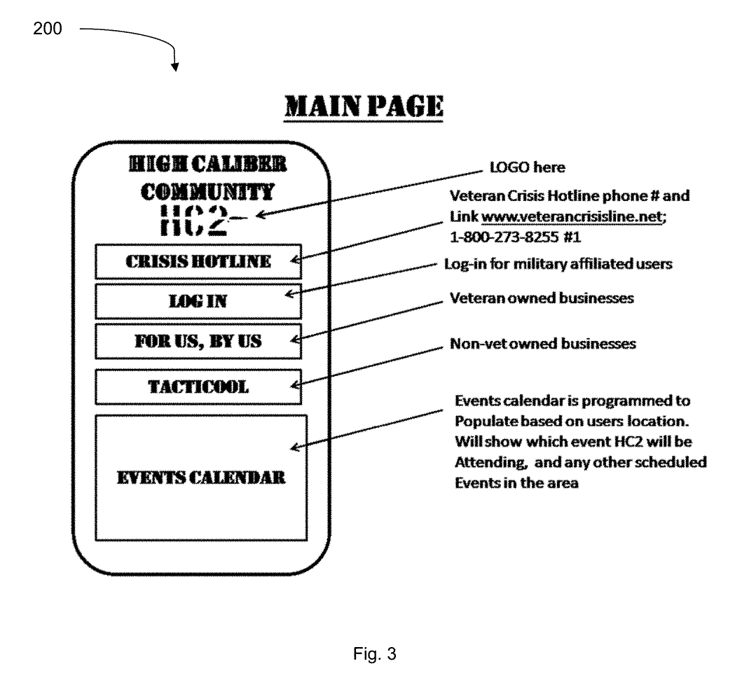

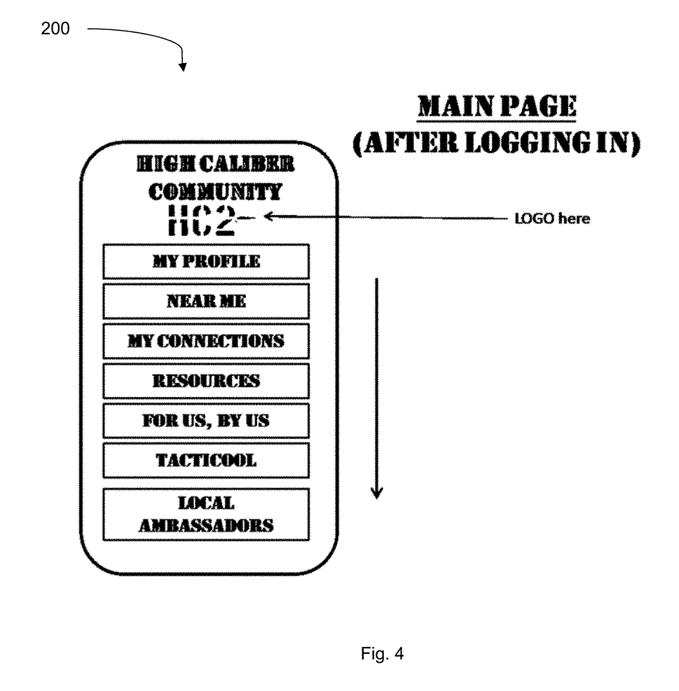

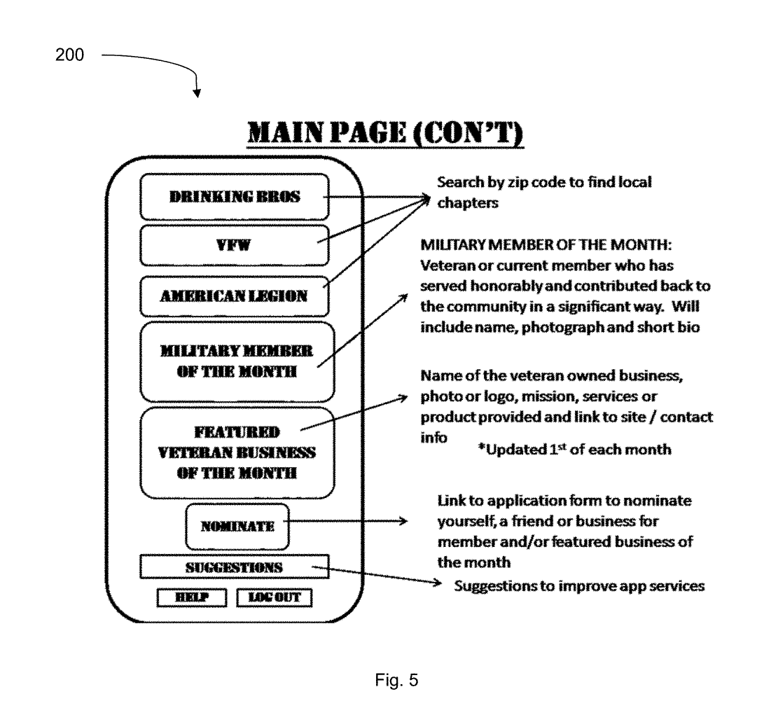

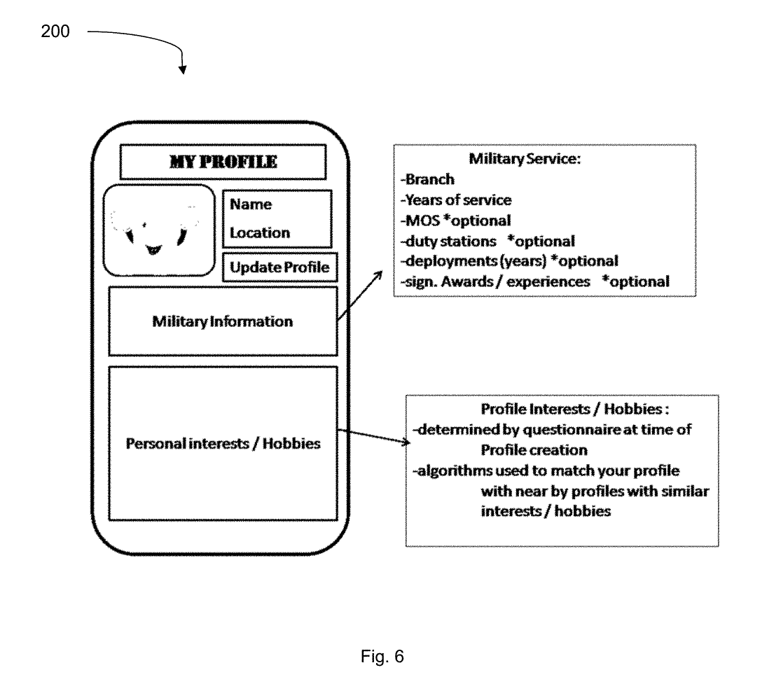

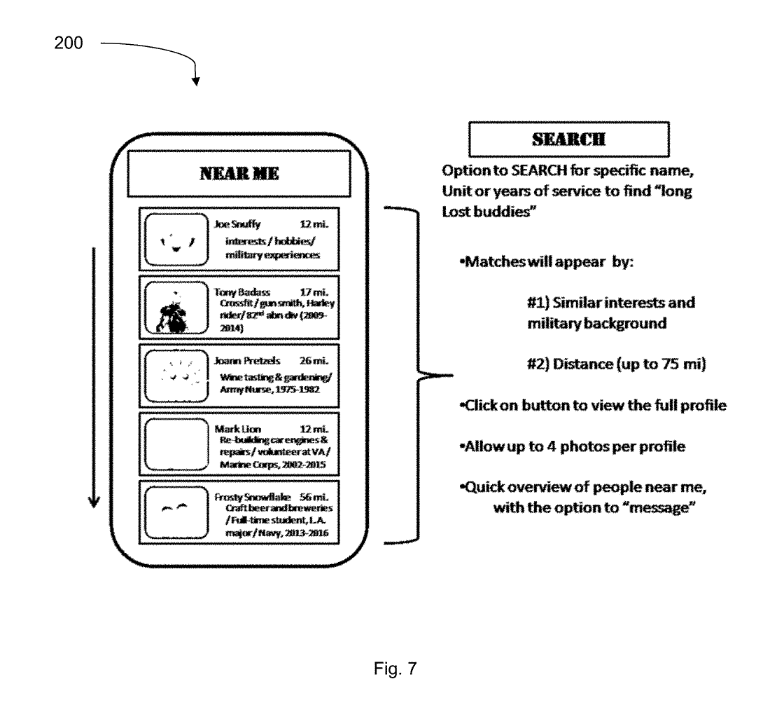

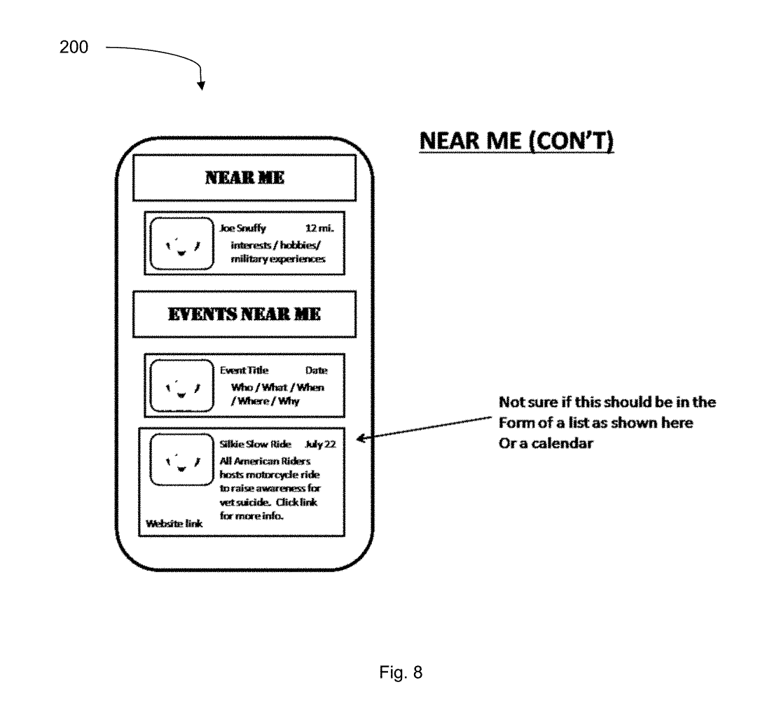

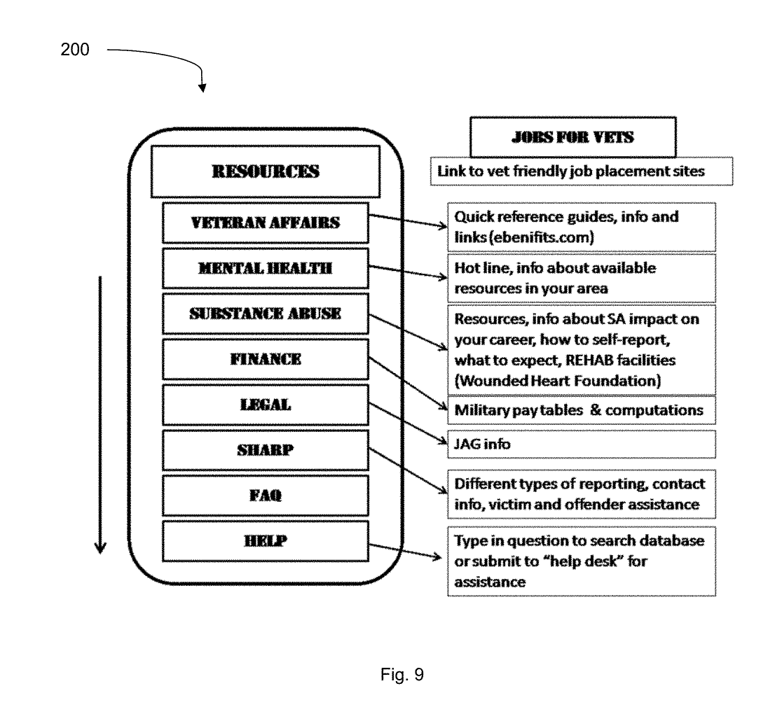

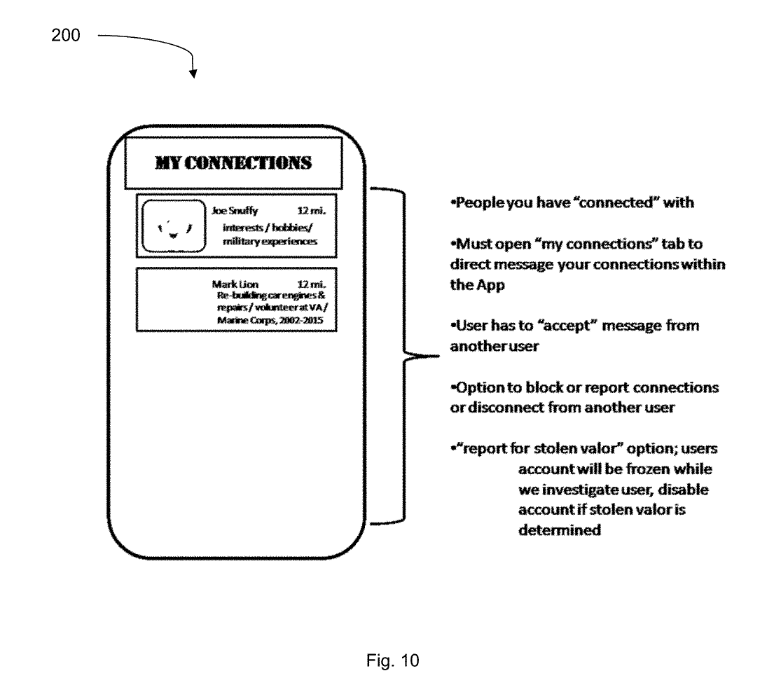

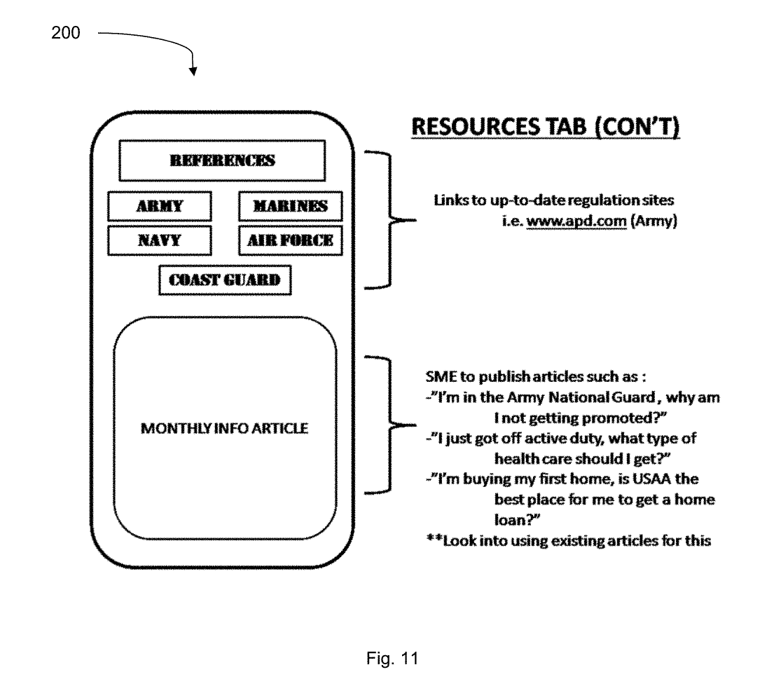

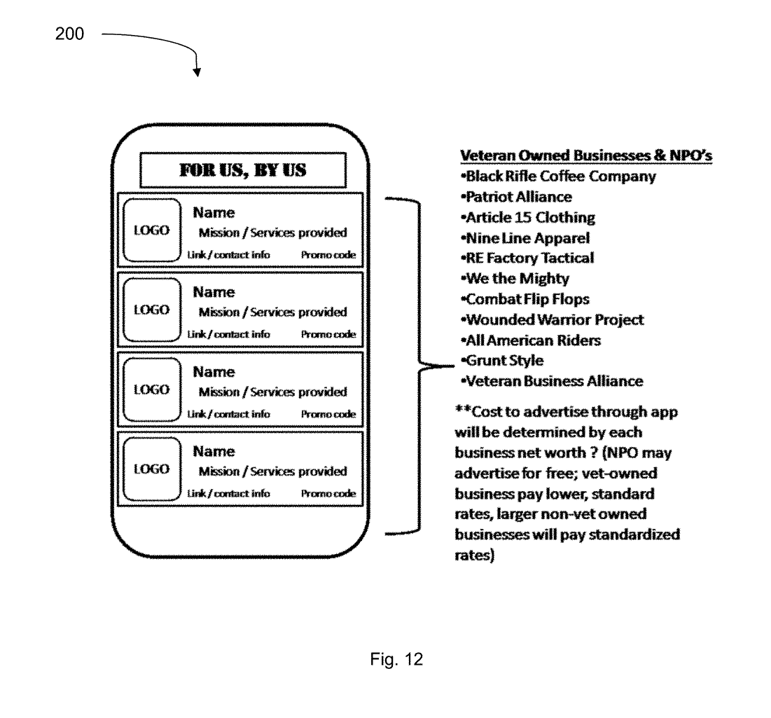

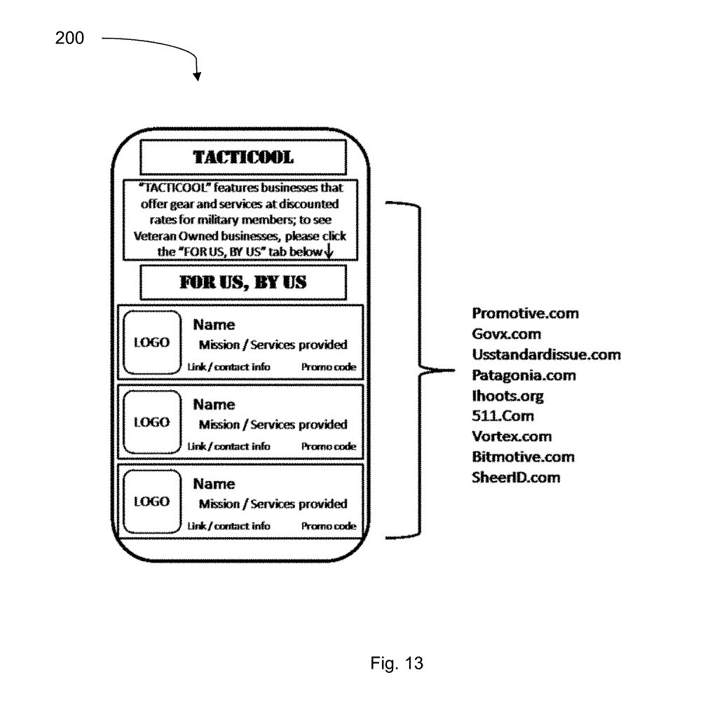

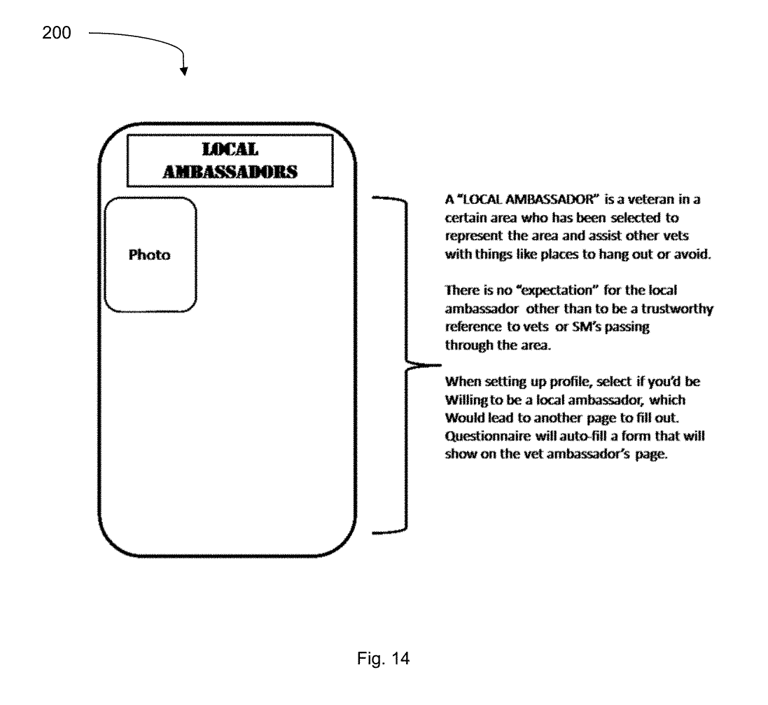

Users interact with social networking system 110 on a client device 120 using a user interface 200. One embodiment of a user interface is illustrated in FIGS. 3-14.

Many modifications and other embodiments of the invention will come to mind to one skilled in the art to which this invention pertains having the benefit of the teachings presented in the foregoing descriptions and the associated drawings. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

1. A method of providing social networking, support, and information to the military community comprising:
providing a social networking system to one or more user;
gathering information about the one or more users' military history;
verifying the users' military affiliation; and
providing information to the one or more users.

2. The method of claim 1 further comprising gathering information about the user's mental health.

3. The method of claim 2 further comprising providing information about mental health resources in the user's geographic area.

4. The method of claim 1 wherein the information comprises information about veteran owned businesses.

5. The method of claim 1 wherein the information comprises information about services available to veterans.

6. The method of claim 1 wherein the information comprises information about local ambassadors in the user's geographic area.

7. The method of claim 1 further comprising gathering information about the one or more users' personal interests.

8. The method of claim 7 further comprising connecting each of the one or more users with other users with similar military histories or personal interests.

9. The method of claim 1 further comprising displaying content created by the user to other users of the social networking system.

10. The method of claim 9 further comprising verifying acceptability of the content created by the user prior to displaying the content on the social networking system.

11. The method of claim 1 further comprising providing a crisis notification system.

12. The method of claim 11 wherein the crisis notification system is configured to allow a user to request peer to peer support from other users of the social networking system.
